(19) United States
(12) Patent Application Publication
(10) Pub. No.: US 2022/0168478 A1
(43) Pub. Date: Jun. 2, 2022

(54) NASAL DRESSINGS AND STENTS

(71) Applicant: DSM IP Assets B.V., Heerlen (NL)

(72) Inventor: Timothy RINGEISEN, Exton, PA (US)

(21) Appl. No.: 17/437,864

(22) PCT Filed: Mar. 20, 2020
(86) PCT No.: PCT/US2020/023749

§ 371 Date: Sep. 10, 2021

Related U.S. Application Data
(60) Provisional application No. 62/822,154, filed on Mar. 22, 2019.

(51) Int. Cl. A61L 31/10; A61L 31/04; A61L 31/16; A61L 31/14; A61L 15/22; A61L 15/32; A61L 15/42; A61L 15/44; A61F 13/12

(52) U.S. Cl. CPC A61L 31/10; A61L 31/044; A61L 31/041; A61L 31/16; A61L 31/146; A61L 15/225; A61L 2300/43; A61L 15/425; A61L 15/44; A61F 13/126; A61L 2300/41; A61L 2300/222; A61L 15/325

(57) ABSTRACT

Disclosed are nasal dressings and nasal stents comprising a collagen foam. Also disclosed are methods of making and using such nasal dressings and nasal stents. In an embodiment, a nasal dressing or nasal stent is formed by a method comprising the steps of: forming an aqueous mixture of from 5 to 25 wt % of acid-soluble collagen and from 75 to 95 wt % of collagen fibers, both based on the total solids content of the aqueous mixture, placing the aqueous mixture into a mold, freeze-drying the aqueous mixture while in the mold, thereby forming a collagen foam, and cross-linking the collagen foam.

### CROSS-REFERENCE TO RELATED APPLICATIONS

This application is an international application claiming priority from U.S. Provisional Application No. 62/822,154, filed 22 Mar. 2019.

### FIELD

The disclosed inventions pertain to nasal dressings and stents, and materials and methods for forming nasal dressings and stents.

### BACKGROUND

Nasal dressings and stents are typically used postoperatively. However, a growing number of applications are occurring prior to any surgical intervention, or as treatment of sinus surgery complications or reoccurrence of chronic sinusitis.

Examples of commercially available nasal dressings or stents are MeroPack® bioresorbable nasal dressings and sinus stents that comprises lyophilized and compressed microfibrillar collagen and esterified hyaluronic acid. Exemplary patent publications are U.S. Pat. Nos. 7,858,107, 8,313,762, and 8,765,715. Further examples are HemoPore® and NasoPore® degradable polyurethane foams from Stryker and drug-loaded degradable polymer stents, such as PROPEL products from Intersect ENT.

Drawbacks related to non-resorbable packing materials and stents is the physical need for removal, which requires an additional visit to the clinic, pain and trauma to the newly healing tissues. Drawbacks to existing resorbable packing materials include the need for painful overpacking so that the materials stay in position, early collapse of gel like or fast resorbing materials, and need for removal of materials that resorb to slowly. Drawbacks to existing resorbable stents includes fragmentation of the device resulting in sharp edges that penetrate or irritate the tissue and need for removal of fragments or entire devices that resorb to slowly.

### SUMMARY

In an embodiment, a nasal dressing or nasal stent is provided for use in treating a wound or inflammation in the nasal or sinus cavity of a patient, the nasal dressing or nasal stent comprising a collagen foam comprising cross-links. The collagen foam comprises cross-linked acid-soluble collagen and collagen fibers. In an embodiment, the collagen foam is formed by lyophilizing an aqueous mixture of collagen fibers and acid-soluble collagen followed by cross-linking.

The acid-soluble collagen portion of the collagen foam may degrade or fragment, resulting in structural collapse that allows clearance of the devices from the paranasal sinuses via blowing of the nose and mucociliary action, whereas the collagen fiber portion may provide skeletal stability and stiffness, retarding structural collapse of the acid-soluble collagen portion and extending the residence time of the nasal dressing or nasal stent.

Further potential advantages include the ability for the collagen foam to possess shapes that lie flat when not subjected to an outside force. This may allow advantages in production, handling, packaging, transportation, or storage.

In an embodiment, a nasal dressing or nasal stent is formed by a method comprising the steps of:
a. forming an aqueous mixture of from 5 to 25 wt % of acid-soluble collagen and from 75 to 95 wt % of collagen fibers, both based on the total solids content of the aqueous mixture,
b. placing the aqueous mixture into a mold,
c. freeze-drying the aqueous mixture while in the mold, thereby forming a collagen foam, and
d. cross-linking the collagen foam.

### BRIEF DESCRIPTION OF THE DRAWINGS

FIG. 1 depicts an embodiment of a nasal stent.

FIG. 2 depicts five further embodiments of a nasal stent.

FIG. 3 depicts two further embodiments of a nasal stent.

### DETAILED DESCRIPTION

The nasal dressing or stent comprises acid-soluble collagen. Acid-soluble collagen is collagen that is in a form that is insoluble in an aqueous liquid having a pH of 6.5 and is soluble in an aqueous liquid having a pH of 4. To solubilize the collagen in solution, the pH is driven down to from 2 to 4. However, once in solution, the pH can be brought up to 6.5 without coming out of solution. The acid-soluble collagen is introduced into the aqueous mixture as particles or a powder.

In an embodiment, the acid-soluble collagen has been processed without the aid of enzymes. Non-enzymatically processed, acid-soluble collagen may be produced by milling a cleaned collagen source material, such as hides or skins. To solubilize the collagen in solution, the pH is driven down to from 2 to 4. However, once in solution, the pH can be brought up to 6.5 without coming out of solution. The acid-soluble collagen is introduced into the aqueous mixture as particles or a powder. Typically, collagen for use in the invention can be obtained from any suitable animal source, for example, bovine, porcine, piscine, ovine, caprine, or other sources. The tropocollagen resulting from non-enzymatic processing is unable to undergo spontaneous fibrillation under physiological conditions.

The nasal dressing or stent comprises collagen fibers. Collagen fibers are fibers of collagen and are insoluble in an aqueous liquid having a pH of 3.5. Preferably, the collagen fibers are native collagen fibers, as opposed to reconstituted collagen fibers. In an embodiment, the collagen fibers have an average length of at least 0.5 mm, at least 1 mm, at least 2 mm, at least 3 mm, at least 4 mm, at least 4.5 mm, or at least 5 mm. In an embodiment, the collagen fibers have an average length of at most 15 mm, at most 12 mm, or at most 10 mm.

The nasal dressing or nasal stent is formed by the process of forming an aqueous mixture of acid-soluble collagen and collagen fibers. The working pH range for an aqueous mixture containing acid-soluble collagen and collagen fibers is typically from 2 to 6.5. In an embodiment, the pH of the aqueous mixture is from 4.5 to 6.5. In an embodiment, the aqueous mixture has a solids content of from 2 to 15 wt %, based on the total weight of the aqueous mixture. Solids content is what is left after removing the liquid components of the aqueous mixture, for instance by lyophilizing the aqueous mixture. In an embodiment, the aqueous mixture is introduced into a mold and lyophilized, thereby forming a collagen foam. This method of forming a collagen foam generally leads to a collagen foam having randomly aligned collagen fibers. Following lyophilization, the collagen foam is cross-linked. In an embodiment, cross-linking is performed without use of chemicals, such as by dehydrothermally cross-linking the collagen foam. In an embodiment, the cross-linking is performed with the use of chemicals, such as glutaraldehyde. The final device may be obtained by cutting the material after cross-linking, such as with a laser.

In an embodiment, the nasal dressing or nasal stent is formed according to the process described in U.S. Pat. No. 8,133,500, which is hereby incorporated by reference in its entirety. In an embodiment, a method of forming a nasal dressing or stent comprises the steps of forming an aqueous mixture of from 5 to 25 wt % of acid-soluble collagen and from 75 to 95 wt % of collagen fibers, both based on the total solids content of the aqueous mixture, pressing the aqueous mixture into a sheet, thereby removing at least some of the water from the aqueous mixture, drying the sheet, and cross-linking the sheet. In an embodiment, a material capable of absorbing moisture, such as a wicking material, is placed between the aqueous mixture and the source of the force that results in pressing of the aqueous mixture. In an embodiment, the pressing is carried out on a hydraulic press. After cross-linking, the sheet may then be rolled or folded so that it exerts pressure on a nasal cavity when positioned inside the nasal cavity of a patient. This method of forming a collagen foam generally leads to a collagen foam having collagen fibers aligned in at least one direction.

In an embodiment, the aqueous mixture comprises at least 5 wt %, 6 wt %, 7 wt %, 8 wt %, 9 wt %, 10 wt %, 11 wt %, 12 wt %, 13 wt %, 14 wt %, or 15 wt % of acid-soluble collagen, based on the total solids content of the aqueous mixture. In an embodiment, the aqueous mixture comprises at most 25 wt %, 22 wt %, 20 wt %, or 15 wt % of acid-soluble collagen, based on the total solids content of the aqueous mixture. In an embodiment, the aqueous mixture comprises at least 75 wt %, 80 wt %, or 85 wt % of collagen fibers, based on the total solids content of the aqueous mixture. In an embodiment, the aqueous mixture comprises at most 95 wt %, 92 wt %, 90 wt %, or 85 wt % of collagen fibers, based on the total solids content of the aqueous mixture.

In an embodiment, the collagen foam comprises at least 5 wt %, 6 wt %, 7 wt %, 8 wt %, 9 wt %, 10 wt %, 11 wt %, 12 wt %, 13 wt %, 14 wt %, or 15 wt % of acid-soluble collagen, based on the total weight of the dry collagen foam. In an embodiment, the collagen foam comprises at most 25 wt %, 22 wt %, 20 wt %, or 15 wt % of acid-soluble collagen, based on the total weight of the dry collagen foam. In an embodiment, the collagen foam comprises at least 75 wt %, 80 wt %, or 85 wt % of collagen fibers, based on the total weight of the dry collagen foam. In an embodiment, the collagen foam comprises at most 95 wt %, 92 wt %, 90 wt %, or 85 wt % of collagen fibers, based on the total weight of the dry collagen foam.

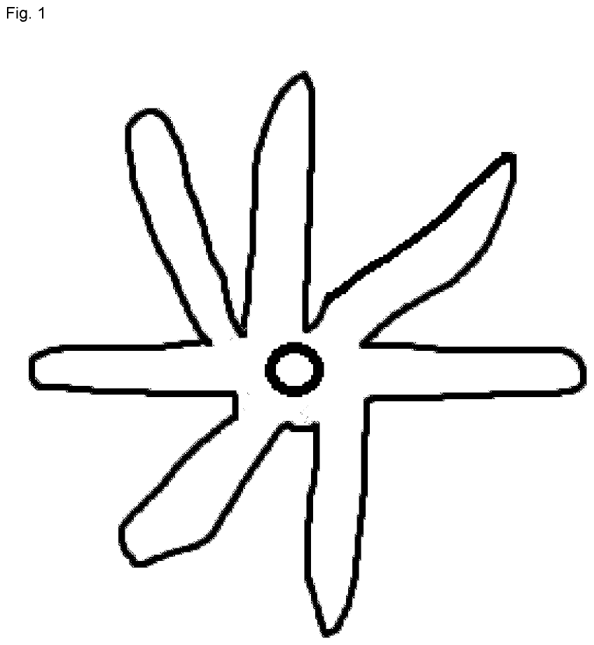

Turning now to FIG. 1, an embodiment of a nasal stent is depicted. The nasal stent comprises a central hub and a plurality of arms radiating from the central hub. The central hub comprises a hole in its center. The hole allows for air passage and can be used in combination with a conical our stepped insertion tool that pushes the sheet into the paranasal sinus and may help with centering the stent in the sinus.

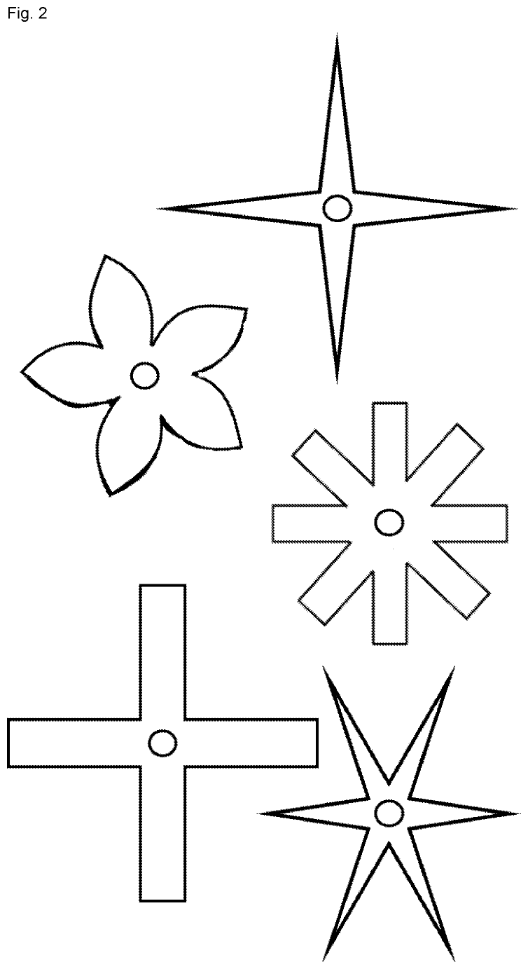

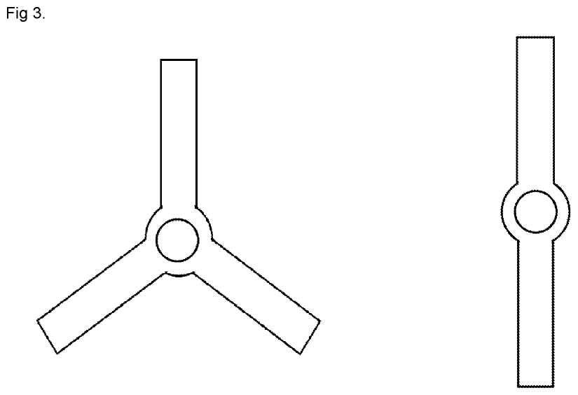

The nasal stent depicted in FIG. 1 may be inserted into the nasal cavity by pushing the stent into the center of a paranasal sinus, causing the radiating arms or spokes to bend backwards towards the entry of the nasal cavity. As the nasal stent attempts to flatten out due its shape memory, it exerts a continuous outward pressure on the paranasal sinus. Further embodiments of nasal stents are depicted in FIGS. 2 and 3.

In an embodiment, the nasal dressing or nasal stent comprises a cross-sectional shape of a circle, cylinder, ellipse or star. In an embodiment, the cross-section of the nasal dressing or stent is in the shape of a partial circle or partial cylinder. In an embodiment, the nasal dressing or stent is in the shape of a cylinder with an opening running down its length. In an embodiment, the nasal dressing or stent is in the shape of a central hub having a plurality of radiating arms. In an embodiment, the nasal dressing or stent is in the shape of a central hub having a plurality of radiating arms and wherein the central hub comprises a hole.

In an embodiment, the nasal dressing or nasal stent comprises an opening throughout its entire length when it is inserted into the nasal cavity of a patient. For example, in each embodiment pictured in FIGS. 1-3, a central hole is present. When the nasal dressing or stent is inserted into the nasal cavity, the radiating arms are bent backwards toward the opening of the nasal cavity. Because there is a hole at the top portion of the nasal dressing or stent, which is the portion of the device inserted furthest into the nasal cavity, and the shape memory of the nasal dressing or stent causes the radiating arms to push outwards, the nasal dressing or stent may be configured such that there is an opening throughout the length of the nasal dressing or stent along the direction of its longitudinal axis. Similarly, such an opening could be formed by providing a nasal dressing or stent in the shape of a sheet, rolling up the sheet such that a central opening is present along its longitudinal axis, and inserting the rolled sheet into the nasal cavity. Such opening may allow for airflow through the nasal cavity.

In an embodiment, the length of the nasal dressing or stent is from 4 cm to 8 cm. In an embodiment, the largest width of the cross-section of the nasal dressing or stent is from 1 to 3 cm.

In an embodiment, the nasal dressing or nasal stent further comprises a therapeutic agent. The therapeutic agent may be, for example, a steroid, an anti-inflammatory agent, or another therapeutic agent that is useful to treat a wound or inflammation in the nasal or sinus cavity of a patient.

The therapeutic may be present in the interior of the collagen foam, such as by soaking the collagen foam in the therapeutic agent or by mixing in microparticles comprising the therapeutic agent when forming the collagen foam. The therapeutic agent could also be present on an exterior surface by forming a coating on the exterior of the collagen foam. The coating may be formed by, for example, dissolving a therapeutic agent in a degradable synthetic polymer and coating the solution on an exterior surface of the nasal dressing or stent. Suitable degradable synthetic polymers may comprise a polylactic acid (PLA), polyglycolic acid (PGA), poly(lactic-co-glycolic acid) (PLGA), polycaprolactone (PCL), polyester amide (PEA) or a combinations thereof or a co-polymer thereof.

The nasal dressing or stent may be retained in the nasal cavity of a patient due to its shape memory. The nasal dressing or stent may be collapsed, such as by rolling up the collagen foam or bending back one or more radiating arms, and inserting the nasal dressing or stent into the nasal cavity of a patient.

In an embodiment, the nasal dressing or nasal stent lies flat when not subjected to any outside force. This property may allow for easier production, transportation, packaging, or storage of the nasal dressing or stent. In an embodiment, the nasal dressing or nasal stent comprises at least one planar face. In an embodiment, the nasal dressing or nasal stent comprises at two planar faces. In an embodiment, the nasal dressing or nasal stent comprises at least one planar face wherein the at least one planar face has the greatest surface area of any face of the nasal dressing or stent. In an embodiment, the nasal dressing or nasal stent comprises at two planar faces and wherein the at least two planar faces are the surfaces with the two greatest surface areas of the nasal dressing or stent.

### Additional Description of Exemplary Embodiments

1. A nasal dressing or nasal stent comprising a collagen foam comprising cross-linked acid-soluble collagen and collagen fibers.
2. A nasal dressing or nasal stent comprising a collagen foam formed from acid-soluble collagen and collagen fibers, wherein the collagen foam comprises cross-links.
3. A nasal dressing or nasal stent comprising a collagen foam comprising cross-links, the collagen foam being formed by lyophilizing an aqueous mixture of collagen fibers and acid-soluble collagen followed by cross-linking.
4. The nasal dressing or nasal stent of any one of the previous exemplary embodiments, for treating a wound or inflammation in the nasal cavity of a patient.
5. The nasal dressing or nasal stent according to any one of the previous exemplary embodiments, wherein the collagen foam comprises acid-soluble collagen and collagen fibers.
6. The nasal dressing or nasal stent according to any one of the previous exemplary embodiments, wherein the collagen foam comprises from 5 to 25 wt % of acid-soluble collagen and from 75 to 95 wt % of collagen fibers.
7. The nasal dressing or nasal stent according to any one of the previous exemplary embodiments, wherein the collagen foam is formed by lyophilizing an aqueous mixture of from 5 to 25 wt % of acid-soluble collagen and from 75 to 95 wt % of collagen fibers, both based on the total solids content of the aqueous mixture, followed by cross-linking.
8. A nasal dressing or nasal stent formed by a method comprising the steps of:
   a. forming an aqueous mixture of from 5 to 25 wt % of acid-soluble collagen and from 75 to 95 wt % of collagen fibers, both based on the total solids content of the aqueous mixture,
   b. placing the aqueous mixture into a mold,
   c. freeze-drying the aqueous mixture while in the mold, thereby forming a collagen foam, and
   d. cross-linking the collagen foam.
9. A method of forming a nasal dressing or nasal stent comprising the steps of:
   a. forming an aqueous mixture of from 5 to 25 wt % of acid-soluble collagen and from 75 to 95 wt % of collagen fibers, both based on the total solids content of the aqueous mixture,
   b. placing the aqueous mixture into a mold,
   c. freeze-drying the aqueous mixture while in the mold, thereby forming a collagen foam, and
   d. cross-linking the collagen foam.
10. A method of forming a nasal dressing or nasal stent comprising the steps of:
   a. forming an aqueous mixture of from 5 to 25 wt % of acid-soluble collagen and from 75 to 95 wt % of collagen fibers, both based on the total solids content of the aqueous mixture,
   b. pressing the aqueous mixture into a sheet, thereby removing at least some of the water from the aqueous mixture,
   c. drying the sheet, and
   d. cross-linking the sheet.
11. The method according to the previous exemplary embodiment, wherein the step of drying the sheet comprises freeze-drying the sheet.
12. A nasal dressing or nasal stent formed according to the method of any one of the previous exemplary embodiments.
13. The method or nasal dressing or nasal stent according to any one of the previous exemplary embodiments, wherein the aqueous mixture has a solids content of from 2 to 15 wt %, based on the total weight of the aqueous mixture.
14. The method or nasal dressing or nasal stent according to any one of the previous exemplary embodiments, wherein the acid-soluble collagen is processed without the use of enzymes.
15. The method or nasal dressing or nasal stent according to any one of the previous exemplary embodiments, wherein the collagen fibers are native collagen fibers.
16. The method or nasal dressing or nasal stent according to any one of the previous exemplary embodiments, wherein the collagen fibers are aligned randomly.
17. The method or nasal dressing or nasal stent according to any one of the previous exemplary embodiments, wherein the collagen fibers are aligned in at least one direction.
18. The method or nasal dressing or nasal stent according to any one of the previous exemplary embodiments, wherein the collagen fibers have an average length of from 1 to 15 mm.
19. The method or nasal dressing or nasal stent according to any one of the previous exemplary embodiments, wherein the collagen fibers have an average length of from 5 to 15 mm.
20. The method or nasal dressing or nasal stent according to any one of the previous exemplary embodiments, wherein the solids content of the aqueous mixture consists of from 5 to 25 wt % of acid-soluble collagen and from 75 to 95 wt % of collagen fibers, both based on the total solids content of the aqueous mixture.
21. The method or nasal dressing or nasal stent according to any one of the previous exemplary embodiments, wherein the aqueous mixture consists of from 5 to 25 wt % of acid-soluble collagen and from 75 to 95 wt % of collagen fibers, both based on the total solids content of the aqueous mixture, and water.
22. The method or nasal dressing or nasal stent according to any one of the previous exemplary embodiments, wherein the aqueous mixture comprises at least 5 wt %, 6 wt %, 7 wt %, 8 wt %, 9 wt %, 10 wt %, 11 wt %, 12 wt %, 13 wt %, 14 wt %, or 15 wt % of acid-soluble collagen, based on the total solids content of the aqueous mixture.
23. The method or nasal dressing or nasal stent according to any one of the previous exemplary embodiments, wherein the aqueous mixture comprises at most 25 wt %, 22 wt %, 20 wt %, or 15 wt % of acid-soluble collagen, based on the total solids content of the aqueous mixture.
24. The method or nasal dressing or nasal stent according to any one of the previous exemplary embodiments, wherein the aqueous mixture comprises at least 75 wt %, 80 wt %, or 85 wt % of collagen fibers, based on the total solids content of the aqueous mixture.
25. The method or nasal dressing or nasal stent according to any one of the previous exemplary embodiments, wherein the aqueous mixture comprises at most 95 wt %, 92 wt %, 90 wt %, or 85 wt % of collagen fibers, based on the total solids content of the aqueous mixture.
26. The method or nasal dressing or nasal stent according to any one of the previous exemplary embodiments, wherein the collagen foam comprises at least 5 wt %, 6 wt %, 7 wt %, 8 wt %, 9 wt %, 10 wt %, 11 wt %, 12 wt %, 13 wt %, 14 wt %, or 15 wt % of acid-soluble collagen, based on the total weight of the dry collagen foam.
27. The method or nasal dressing or nasal stent according to any one of the previous exemplary embodiments, wherein the collagen foam comprises at most 25 wt %, 22 wt %, 20 wt %, or 15 wt % of acid-soluble collagen, based on the total weight of the dry collagen foam.
28. The method or nasal dressing or nasal stent according to any one of the previous exemplary embodiments, wherein the collagen foam comprises at least 75 wt %, 80 wt %, or 85 wt % of collagen fibers, based on the total weight of the dry collagen foam.
29. The method or nasal dressing or nasal stent according to any one of the previous exemplary embodiments, wherein the collagen foam comprises at most 95 wt %, 92 wt %, 90 wt %, or 85 wt % of collagen fibers, based on the total weight of the dry collagen foam.
30. The method or nasal dressing or nasal stent according to the previous exemplary embodiment, wherein the aqueous mixture comprises from 8 to 20 wt % of acid-soluble collagen and from 80 to 92 wt % of collagen fibers.
31. The method or nasal dressing or nasal stent according to any one of the previous exemplary embodiments, wherein the solids content of the aqueous mixture consists of from 8 to 20 wt % of acid-soluble collagen and from 80 to 92 wt % of collagen fibers, both based on the total solids content of the aqueous mixture, and water.
32. The method or nasal dressing or nasal stent according to any one of the previous exemplary embodiments, wherein the solids content of the aqueous mixture consists of from 8 to 20 wt % of acid-soluble collagen and from 80 to 92 wt % of collagen fibers, both based on the total solids content of the aqueous mixture, and water.
33. The method or nasal dressing or nasal stent according to any one of the previous exemplary embodiments, further comprising the step of cutting the cross-linked collagen foam into a desired shape.
34. The method or nasal dressing or nasal stent according to any one of the previous exemplary embodiments, further comprising the step of laser cutting the cross-linked collagen foam into a desired shape.
35. The method or nasal dressing or nasal stent according to any one of the previous exemplary embodiments, wherein the nasal dressing or nasal stent comprises an opening throughout its entire length when the nasal dressing or nasal stent is present in the nasal cavity of a patient.
36. The method or nasal dressing or nasal stent according to any one of the previous exemplary embodiments, wherein the nasal dressing or nasal stent comprises an opening throughout the length of the nasal dressing or stent along the direction of its longitudinal axis when the nasal dressing or stent is positioned in the nasal cavity of a patient.
37. The method or nasal dressing or nasal stent according to any one of the previous exemplary embodiments, wherein the cross-sectional shape of the nasal dressing or nasal stent is a circle, cylinder, ellipse or star.
38. The method or nasal dressing or nasal stent according to any one of the previous exemplary embodiments, wherein the cross-section of the nasal dressing or stent comprises the shape of a partial circle or partial cylinder.
39. The method or nasal dressing or nasal stent according to any one of the previous exemplary embodiments, wherein the nasal dressing or stent comprises the shape of a cylinder with an opening running down its length.
40. The method or nasal dressing or nasal stent according to any one of the previous exemplary embodiments, wherein the nasal dressing or stent comprises the shape of a central hub having a plurality of radiating arms.
41. The method or nasal dressing or nasal stent according to any one of the previous exemplary embodiments, wherein the nasal dressing or stent comprises the shape of a central hub having a plurality of radiating arms and wherein the central hub comprises a hole.
42. The method or nasal dressing or nasal stent according to any one of the previous exemplary embodiments, wherein the length of the nasal dressing or stent is from 4 cm to 8 cm.
43. The method or nasal dressing or nasal stent according to any one of the previous exemplary embodiments, wherein the largest width of the cross-section of the nasal dressing or stent is from 1 to 3 cm.
44. The method or nasal dressing or nasal stent according to any one of the previous exemplary embodiments, further comprising a therapeutic agent.
45. The method or nasal dressing or nasal stent according to any one of the previous exemplary embodiments, wherein the therapeutic agent comprises a steroid.
46. The method or nasal dressing or nasal stent according to any one of the previous exemplary embodiments, wherein the therapeutic agent comprises an anti-inflammatory agent.
47. The method or nasal dressing or nasal stent according to any one of the previous exemplary embodiments, further comprising a therapeutic agent present in the interior of the nasal dressing or stent.
48. The method or nasal dressing or nasal stent according to any one of the previous exemplary embodiments, further comprising a coating comprising a therapeutic agent present on an exterior surface of the nasal dressing or stent.
49. The method or nasal dressing or nasal stent according to any one of the previous exemplary embodiments, further comprising a coating comprising a therapeutic agent and a degradable synthetic polymer present on an exterior surface of the nasal dressing or stent.
50. The method or nasal dressing or nasal stent according to any one of the previous exemplary embodiments, further comprising a therapeutic agent and a degradable synthetic polymer present on an exterior surface of the nasal dressing or stent.
51. The method or nasal dressing or nasal stent according to any one of the previous exemplary embodiments, wherein the degradable synthetic polymer comprises a polylactic acid (PLA), polyglycolic acid (PGA), poly(lactic-co-glycolic acid) (PLGA), polycaprolactone (PCL), polyester amide (PEA) or a combinations thereof or a co-polymer thereof.
52. The method or nasal dressing or nasal stent according to any one of the previous exemplary embodiments, further comprising a therapeutic agent present throughout the nasal dressing or stent.
53. The method according to any one of the previous exemplary embodiments, further comprising the step of soaking into the nasal dressing or stent a composition comprising a therapeutic agent dissolved in a degradable polymer.
54. The method according to any one of the previous exemplary embodiments, further comprising the step of coating on the exterior surface of the nasal dressing or stent a composition comprising a therapeutic agent dissolved in a degradable polymer.
55. The method according to any one of the previous exemplary embodiments, further comprising the step of freeze-drying the nasal dressing or stent.
56. A method of treating a wound comprising the step of inserting the nasal dressing or nasal stent according to any one of the previous exemplary embodiments into the nasal cavity of a patient.
57. The method of treating a wound according to any one of the previous exemplary embodiments, further comprising the step of collapsing the nasal dressing or nasal stent prior to inserting the nasal dressing or nasal stent in the nasal cavity.
58. The method of treating a wound according to any one of the previous exemplary embodiments, wherein the nasal dressing or nasal stent automatically expands toward its original shape after being inserted into the nasal cavity.
59. The method or nasal dressing or nasal stent according to any one of the previous exemplary embodiments, wherein the nasal dressing or nasal stent comprises shape memory.
60. The method or nasal dressing or nasal stent according to any one of the previous exemplary embodiments, wherein the nasal dressing or nasal stent comprises shape memory and the shape memory serves to exert outward pressure on the nasal cavity of a patient.
61. The method or nasal dressing or nasal stent according to any one of the previous exemplary embodiments, wherein the nasal dressing or nasal stent lies flat when not subjected to any outside force.
62. The method or nasal dressing or nasal stent according to any one of the previous exemplary embodiments, wherein the nasal dressing or nasal stent comprises at least one planar face.
63. The method or nasal dressing or nasal stent according to any one of the previous exemplary embodiments, wherein the nasal dressing or nasal stent comprises at two planar faces.
64. The method or nasal dressing or nasal stent according to any one of the previous exemplary embodiments, wherein the nasal dressing or nasal stent comprises at least one planar face wherein the at least one planar face has the greatest surface area of any face of the nasal dressing or stent.
65. The method or nasal dressing or nasal stent according to any one of the previous exemplary embodiments, wherein the nasal dressing or nasal stent comprises at least one planar face wherein the at least one planar face has the greatest surface area of any surface of the nasal dressing or stent.
66. The method or nasal dressing or nasal stent according to any one of the previous exemplary embodiments, wherein the nasal dressing or nasal stent comprises at two planar faces and wherein the at least two planar faces are the surfaces with the two greatest surface areas of the nasal dressing or stent.
67. The use of the nasal dressing or nasal stent for treating a wound or inflammation in the nasal cavity of a patient.
68. The nasal dressing or nasal stent of any one of the previous exemplary embodiments for treating a wound or inflammation in the nasal cavity of a patient.
69. A method of treating a patient having a wound or inflammation in the nasal or sinus cavity comprising the step of inserting the nasal dressing or nasal stent of any one of the previous exemplary embodiments into the nasal cavity of a patient.
70. A method of treating a patient having a wound or inflammation in the nasal or sinus cavity comprising the step of collapsing the nasal dressing or nasal stent of any one of the previous exemplary embodiments and inserting the nasal dressing or nasal stent into the nasal cavity of a patient.
71. A method of treating a patient having a wound or inflammation in the nasal or sinus cavity comprising the step of collapsing the nasal dressing or nasal stent of any one of the previous exemplary embodiments and inserting the nasal dressing or nasal stent into the nasal cavity of a patient.
72. A method of treating a patient having a wound or inflammation in the nasal or sinus cavity comprising the steps of providing the nasal dressing or nasal stent of any one of the previous exemplary embodiments, bending the nasal dressing or nasal stent such that the nasal dressing or stent exerts outward pressure due to its shape memory, and inserting the nasal dressing or nasal stent into the nasal cavity of a patient.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. While certain optional features are described as embodiments of the invention, the description is meant to encompass and specifically disclose all combinations of these embodiments unless specifically indicated otherwise or physically impossible.

1. A nasal dressing or nasal stent for treating a wound or inflammation in the nasal cavity of a patient comprising a collagen foam comprising cross-linked acid-soluble collagen and collagen fibers, wherein the nasal dressing or stent comprises the shape of a central hub having a plurality of radiating arms.

2. The nasal dressing or nasal stent according to claim 1, wherein the collagen foam comprises from 5 to 25 wt % of acid-soluble collagen and from 75 to 95 wt % of collagen fibers.

3. The nasal dressing or nasal stent according to claim 1, wherein the collagen foam is formed by lyophilizing an aqueous mixture of from 5 to 25 wt % of acid-soluble collagen and from 75 to 95 wt % of collagen fibers, both based on the total solids content of the aqueous mixture, followed by cross-linking.

4. The nasal dressing or stent according to claim 1, wherein the nasal dressing or nasal stent is formed by a method comprising the steps of:
a. forming an aqueous mixture of from 5 to 25 wt % of acid-soluble collagen and from 75 to 95 wt % of collagen fibers, both based on the total solids content of the aqueous mixture,
b. placing the aqueous mixture into a mold,
c. freeze-drying the aqueous mixture while in the mold, thereby forming a collagen foam, and
d. cross-linking the collagen foam.

5. The nasal dressing or stent according to claim 1, wherein the nasal dressing or nasal stent is formed by a method comprising the steps of:
a. forming an aqueous mixture of from 5 to 25 wt % of acid-soluble collagen and from 75 to 95 wt % of collagen fibers, both based on the total solids content of the aqueous mixture,
b. pressing the aqueous mixture into a sheet, thereby removing at least some of the water from the aqueous mixture,
c. drying the sheet, and
d. cross-linking the sheet.

6. The nasal dressing or nasal stent according claim 2, wherein the collagen fibers are native collagen fibers.

7. The nasal dressing or nasal stent according to claim 6, wherein the collagen fibers have an average length of from 1 to 15 mm.

8. (canceled)

9. (canceled)

10. The nasal dressing or nasal stent according to claim 1, wherein the central hub comprises a hole.

11. (canceled)

12. (canceled)

13. The nasal dressing or nasal stent according to claim 1, wherein the nasal dressing or stent exerts outward pressure on the nasal cavity of a patient when positioned within the nasal cavity.

14. The nasal dressing or nasal stent according to claim 1, wherein the nasal dressing or nasal stent lies flat when not subjected to any outside force.

15. The nasal dressing or nasal stent according to claim 1, wherein the nasal dressing or nasal stent comprises at least one planar face.

16. The nasal dressing or nasal stent according to claim 1, wherein the nasal dressing or nasal stent comprises at two planar faces.

17. The nasal dressing or nasal stent according to claim 1, wherein the nasal dressing or nasal stent comprises at least one planar face wherein the at least one planar face has the greatest surface area of any face of the nasal dressing or stent.

18. The nasal dressing or nasal stent according to claim 1, wherein the nasal dressing or nasal stent comprises at two planar faces and wherein the at least two planar faces are the surfaces with the two greatest surface areas of the nasal dressing or stent.

19. The nasal dressing or nasal stent according to claim 1, further comprising a therapeutic agent.

20. The nasal dressing or nasal stent according to claim 19, wherein the therapeutic agent comprises a steroid or an anti-inflammatory agent.

21. (canceled)

22. The nasal dressing or nasal stent according to claim 1, further comprising a therapeutic agent present in the interior of the nasal dressing or stent.

23. The nasal dressing or nasal stent according to claim 1, further comprising a coating comprising a therapeutic agent present on an exterior surface of the nasal dressing or stent.

24. The nasal dressing or nasal stent according to claim 1, further comprising a therapeutic agent and a degradable synthetic polymer present on an exterior surface of the nasal dressing or stent.

25. The nasal dressing or nasal stent according to claim 24, wherein the degradable synthetic polymer comprises a polylactic acid (PLA), polyglycolic acid (PGA), poly(lactic-co-glycolic acid) (PLGA), polycaprolactone (PCL), polyester amide (PEA) or a combinations thereof or a co-polymer thereof.
